(19) United States
(12) Patent Application Publication
(10) Pub. No.: US 2015/0259614 A1
(43) Pub. Date: Sep. 17, 2015

(54) METHOD AND DEVICE FOR MANUFACTURING BIODIESEL FROM SEWAGE SLUDGE

(71) Applicant: Korea University Research and Business Foundation, Seoul (KR)

(72) Inventors: Jae Woo Lee, Seoul (KR); Ou-Kyung Choi, Yongin (KR)

(21) Appl. No.: 14/668,508

(22) Filed: Mar. 25, 2015

Related U.S. Application Data
(63) Continuation of application No. PCT/KR2014/002972, filed on Apr. 7, 2014.

(30) Foreign Application Priority Data
May 15, 2013 (KR) 10-2013-0055031

(51) Int. Cl. C10L 1/02; C07C 67/02; C07C 67/58; B01J 19/24

(52) U.S. Cl. CPC C10L 1/026; B01J 19/24; C07C 67/02; C07C 67/58; C10L 2200/0476; C10L 2270/026; C10L 2290/54; C10L 2290/06; B01J 2219/24

(57) ABSTRACT

Disclosed are a method and an apparatus for producing biodiesel from water-containing sewage sludge, using xylene or toluene, higher in boiling point than water, as a cosolvent.

### CROSS-REFERENCE TO RELATED APPLICATIONS

This application is a continuation of International Application No. PCT/KR2014/002972 filed on Apr. 7, 2014, which claims priority to Korean Patent Application No. 10-2013-0055031, filed on May 15, 2013. The applications are incorporated herein by reference.

### TECHNICAL FIELD

The present invention relates to a method and an apparatus for the production of biodiesel from sewage sludge. More particularly, the present invention relates to a method and an apparatus for producing biodiesel by transesterifying oil components and fatty acids of primary and secondary sewage sludge generated upon sewage treatment.

### BACKGROUND ART

In South Korea, the production of sludge during sewage treatment amounts to approximately 9,000 tons/day and increases every year. However, the direct landfill of sewage sludge has been prohibited when its water content is 85% or higher, and ocean disposal, a main treatment method, also has been banned from 2012. Accordingly, there is an urgent need for a technical method for stably treating and disposing sewage sludge. Meanwhile, keen attention has been paid to energy conversion techniques from waste resources such as sewage, with an increase in the international awareness of energy security and climate change.

Anaerobic digestion is a currently used inland treatment technique for sewage sludge which is arousing great interest. However, the anaerobic digestion technique is known to suffer from the disadvantages of: being poor in digestion rate due to long chain fatty acids in the waste activated sludge (secondary sludge), consuming high energy for drying sludge in conversion into a fuel, and producing a foul odor. Meanwhile, an active attempt has been made, particularly in the USA to convert the oil components of primary and secondary sewage sludge and the fatty acids, used as phospholipid membranes of microorganisms, of activated sludge, into biodiesel by transesterification. When produced from organic wastes such as sewage sludge, biodiesel has an advantage over biogas in terms of the easy of storage and handling. Further, the production of biodiesel from sewage sludge is very valuable in terms of renewable energy production as well as resource recycling, with the subsequent propagation of an enormous ripple effect. Biodiesel is now regarded as an important alternative to traditional petroleum diesel. The Ministry of Trade, Industry and Energy in South Korea established the goal of increasing the domestic distribution rate up to 7% by 2030. For conventional biodiesel production methods, however, a very high proportion of the cost of material is in the production cost. Particularly, it is very difficult to generate plant-based raw materials in South Korea because its arable land resources are limited. For these reasons, most of the biodiesel consumed in South Korea is imported. Accordingly, the use of sewage sludge as a raw material for new biodiesel is very important and valuable in terms of energy production in lieu of energy importation and the recycling management of waste resources that are difficult to dispose of.

Studies on esterification technology to produce biodiesel from sewage sludge still remain in a nascent stage, and most of them are limited to the use of dried sewage sludge as a material.

### SUMMARY

The present invention aims to produce biodiesel with a high content of fatty acid methyl ester from sewage sludge by transesterification using a hydrophobic cosolvent having a high boiling point.

Also, the present invention aims to enhance the efficiency of the esterification reaction, useful for the production of biodiesel from sewage sludge, by separating oils and fatty acids from water of the sewage via proper temperature control, and reacting the oils and fatty acids with methanol.

To accomplish above objects, the present invention provides a method for producing biodiesel, comprising:

(a) heating water-containing sewage sludge and a mixture solution, together with xylene or toluene as a cosolvent, in a reaction tank 30 to induce a transesterification reaction, the mixture solution including methanol or ethanol and an acid catalyst at a volume ratio of 1:0.01 to 1:0.05;

(b) cooling and condensing a mixed steam generated in step (a) to separate respective layers of an aqueous methanol or ethanol solution and the cosolvent, followed by circulating the cosolvent to the reaction tank;

(c) further adding methanol and ethanol to the reaction tank to perform the transesterification reaction to completion, with the concomitant production of biodiesel; and

(d) isolating and recovering the biodiesel from the aqueous methanol or ethanol solution, the cosolvent, and the residual solid.

Also, the present invention provides an apparatus for producing biodiesel, comprising:

a cosolvent reservoir 10;

a solvent reservoir 20;

a reaction tank 30, provided at upper positions thereof with a solvent supply unit 90 for introducing a cosolvent and a solvent, respectively, from the cosolvent reservoir and the solvent reservoir and with a sludge supply unit 80, at a lower portion with a sludge discharge unit 120 for discharging residual sludge from the reactor tank, at a middle position with a biodiesel discharge unit 110, and at a top position with a water steam discharge unit 100;

a condenser connected to the water steam discharge unit 40;

a liquid phase separator 50 for separating the condensed, aqueous methanol or ethanol solution that is released from the condenser;

a solvent recovery unit 41 for recovering the condensed solvent discharged from the condenser;

a solvent recovery tank 60 for storing the solvent recovered by the solvent recovery unit 41; and

a solvent circulating pump 70.

Characterized by using xylene or toluene, high in boiling point, as a cosolvent, the method for producing biodiesel from sewage sludge allows the transesterification to proceed without water inhibition, bringing about the advantage of decreasing the amount of methanol to be used, and improving the conversion of fatty acid methyl esters from lipids or fatty acids to produce biodiesel with a high content of fatty acid methyl esters at a high yield.

### DESCRIPTION OF DRAWINGS

FIG. 1 is a schematic view of an apparatus for producing biodiesel in accordance with an exemplary embodiment of the present invention.

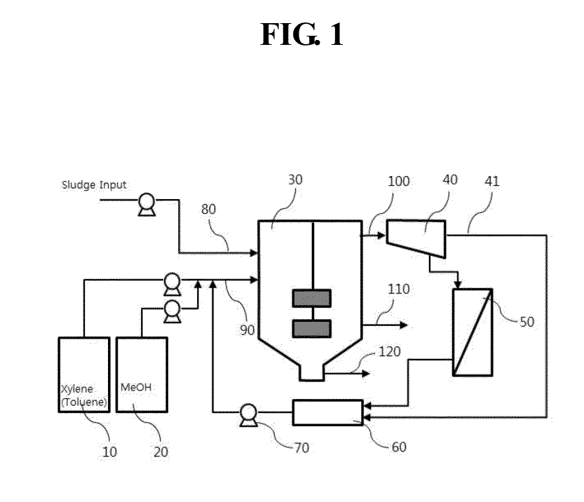

(10: cosolvent reservoir, 20: solvent reservoir, 30: reaction tank, 40: condenser, 41: solvent recovery unit, 50: liquid phase separator, 51: conveyor, 52: potassium carbonate supply pump, 53: methanol or ethanol recovering unit, 54: water discharging unit, 60: solvent recovery tank, 70: solvent circulating pump, 80: sludge supply unit, 90: solvent supply unit, 100: water steam discharge unit, 110: biodiesel discharge unit, 120: sludge discharge unit).

FIG. 2 is a schematic view of a condenser in which a cosolvent is separated from methanol or ethanol.

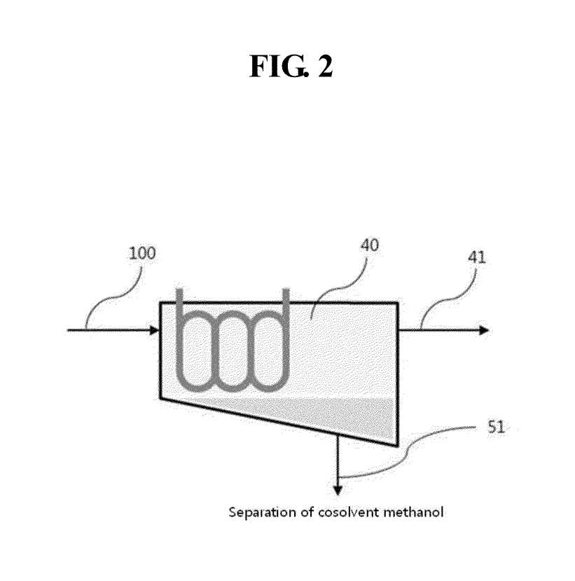

FIG. 3 is a schematic view of a liquid phase separator in which methanol or ethanol is separated from water.

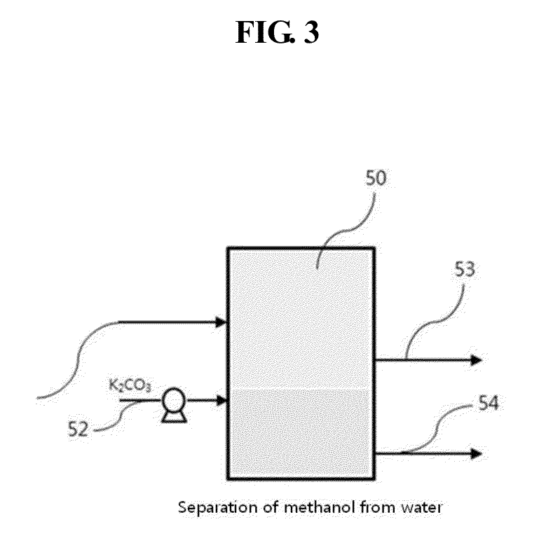

FIG. 4 is a mass spectrum showing fatty acid methyl esters of Example 1.

FIG. 5 is a mass spectrum showing fatty acid methyl esters of Comparative Example 1.

### DETAILED DESCRIPTION

Below, a detailed description will be given of the present invention.

The present invention addresses a method for producing biodiesel from sewage sludge. The method includes:

(a) heating water-containing sewage sludge and a mixture solution, together with xylene or toluene as a cosolvent, in a reaction tank to induce a transesterification reaction, the mixture solution including methanol or ethanol and an acid catalyst at a volume ratio of 1:0.01 to 1:0.05;

(b) cooling and condensing the mixed steam generated in step (a) to separate respective layers of an aqueous methanol or ethanol solution and the cosolvent, followed by circulating the cosolvent to the reaction tank;

(c) further adding methanol and ethanol to the reaction tank to perform the transesterification reaction to completion, with the concomitant production of biodiesel; and

(d) isolating and recovering the biodiesel from the aqueous methanol or ethanol solution, the cosolvent, and the residual solid.

The sewage sludge may be primary or secondary sewage sludge, or may be before a drying process has been carried out. The water content of sewage sludge may be preferably in the order of 0 to 85% by weight, or more preferably in the order of 80 to 85% by weight.

As a solvent for transesterification, methanol or ethanol may be used. An acid catalyst is added to the solvent to increase an initial reaction rate. Preferably, the volume ratio of methanol or ethanol to acid catalyst ranges from 1:0.01 to 1:0.05. Within the range, biodiesel can be produced at a high yield.

In addition, the acid catalyst may be selected from the group consisting of, but not limited to, sulfuric acid, hydrochloric acid, sulfonic acid, and a combination thereof, with the most preference for sulfuric acid.

Xylene or toluene may function as the cosolvent. Because the cosolvents boil at 138.5 and 111° C., respectively, water can be separated in the transesterification reaction. That is, when being heated, the water of the sludge is evaporated and discharged before the temperature reaches the boiling points of the hydrophobic cosolvent. In the meanwhile, hydrophobic oils and fatty acids migrate to and remain in the hydrophobic cosolvent to increase the production yield of biodiesel and the content of fatty acid methyl esters and to decrease the amount of methanol to be used.

The mixture solution of methanol or ethanol and acid catalyst may be preferably mixed at a volume ratio of 1:0.25 to 1:1 with the cosolvent, and more preferably at a volume ratio of 1:0.5 to 1:0.75. At the preferable volume ratio between methanol and the cosolvent, the hydrophobic oil and fatty acid components extracted by transesterification can migrate into the cosolvent at maximum efficiency.

In addition, the transesterification reaction may be preferably carried out at 80 to 110° C. for 4 to 8 hrs and more preferably at 90 to 105° C. In the temperature range, the time of transesterification can be reduced by 4 hrs to 8 hrs, compared to that using a low-boiling point cosolvent such as hexane.

The mixed gas generated during the transesterification of step (a) includes water vapor, methanol or ethanol gas, and cosolvent gas, and is cooled and condensed in a condenser 40. At this time, layer separation occurs into an aqueous methanol or ethanol solution layer and a cosolvent layer. The separated cosolvent is circulated back to the reaction tank.

Afterwards, the further addition of methanol or ethanol to the reaction tank leads the transesterification to the completion. The methanol or ethanol solution separated in step (b) is introduced into a liquid phase separator to which a salt is then added to separate methanol or ethanol from water. This methanol or ethanol may be used. That is, it can be recycled to the reaction tank where the transesterification reaction is completed. The salt may be calcium carbonate or sodium carbonate, but is not limited thereto.

Step (d) is to separate and recover the produced biodiesel from the methanol or ethanol, the cosolvent and the residues by gravitational sedimentation after the completion of transesterification. For effective separation, water may be further added.

In the produced biodiesel of step (c), the cosolvent is still included. Thus, separation of the cosolvent is additionally needed so as to increase the purity of biodiesel. By conducting steps (a) to (d), biodiesel with a high content of fatty acid methyl esters can be produced at high yield.

Also, the present invention addresses an apparatus for producing biodiesel. The apparatus includes a cosolvent reservoir 10; a solvent reservoir 20; a reaction tank 30 provided at upper positions thereof with a solvent supply unit 90 for introducing a cosolvent and a solvent respectively from the cosolvent reservoir 10 and the solvent reservoir 20 and with a sludge supply unit 80, at a lower portion with a sludge discharge unit 120 for discharging residual sludge from the reactor tank, at a middle position with a biodiesel discharge unit 110, and at a top position with a water steam discharge unit 100; a condenser 40 connected to the water steam discharge unit; a liquid phase separator 50 for separating the condensed, aqueous methanol or ethanol solution that is released from the condenser; a solvent recovery unit 41 for recovering the condensed solvent discharged from the condenser; a solvent recovery tank 60; and a solvent circulating pump 70.

In one exemplary embodiment of the present invention, the aqueous methanol or ethanol solution discharged from the condenser 40 is transferred to the liquid phase separator 50 by a conveyor 51. Introduction of potassium carbonate into the liquid phase separator by a potassium carbonate supply pump 52 causes the aqueous methanol or ethanol solution to be separated into water and methanol or ethanol. The water is discharged to a water discharging unit 54 while the methanol or ethanol is flowed towards the solvent recovery tank 60 by a methanol or ethanol recovering unit 53.

### EXAMPLES

A better understanding of the present invention may be obtained through the following examples which are set forth to illustrate, but are not to be construed as limiting the present invention.

### Example 1

### Production of Biodiesel from Sewage Sludge Using Xylene

To a flask was introduced 150 g of sewage sludge with a water content of 85% by weight. Then, a mixture solution of 300 mL of methanol and 15 mL of sulfuric acid was added, together with 300 mL of xylene, to the flask. After the secondary sewage sludge was completely dissolved in the solvent mixture, transesterification was carried out at 105° C. for 4 hrs. The mixed steam generated during heating was cooled in a condenser to form two separate layers of xylene and an aqueous methanol solution. After being isolated, the aqueous methanol solution was further separated into water and methanol using potassium carbonate. Both the methanol and the xylene, thus separated, were recycled to the reaction tank 30. When the transesterification was completed, water was added to the reaction tank to allow the separation of biodiesel from the aqueous methanol solution and residual solids by gravitational sedimentation.

### Comparative Example 1

### Production of Biodiesel from Sewage Sludge Using Hexane

The same procedure as in Example 1 was repeated, with the exception that hexane was used instead of xylene and the transesterification was carried out for 4 hrs at 55° C. instead of at 105° C.

### Experimental Example 1

### Production Yield of Biodiesel from Sewage Sludge

The production yields of biodiesel in Example 1 and Comparative Example 1 were measured, and the results are summarized in Table 1, below.

TABLE 1

| | Rxn. Temp. | Rxn. Time | Biodiesel Yield |
|---|---|---|---|
| Solvent | (° C.) | (hr) | (%) |
| Ex. 1 (Xylene) | 105 | 4 | 15.9 |
| C. Ex. 1(Hexane) | 55 | 8 | 8.22 |

In Comparative Example 1 where hexane having a boiling point of 69° C. (lower than that of water) was employed as a solvent, a low production yield of biodiesel was obtained. In contrast, the production yield of biodiesel in Example 1 using xylene, which boils at a higher temperature than does water, amounted to as high as 15.9%. Because it was difficult to extract the water of sewage from the biodiesel when the boiling point of the solvent was lower than that of water, the reaction could not be easily carried out, and thus the production of biodiesel from sewage was inefficient.

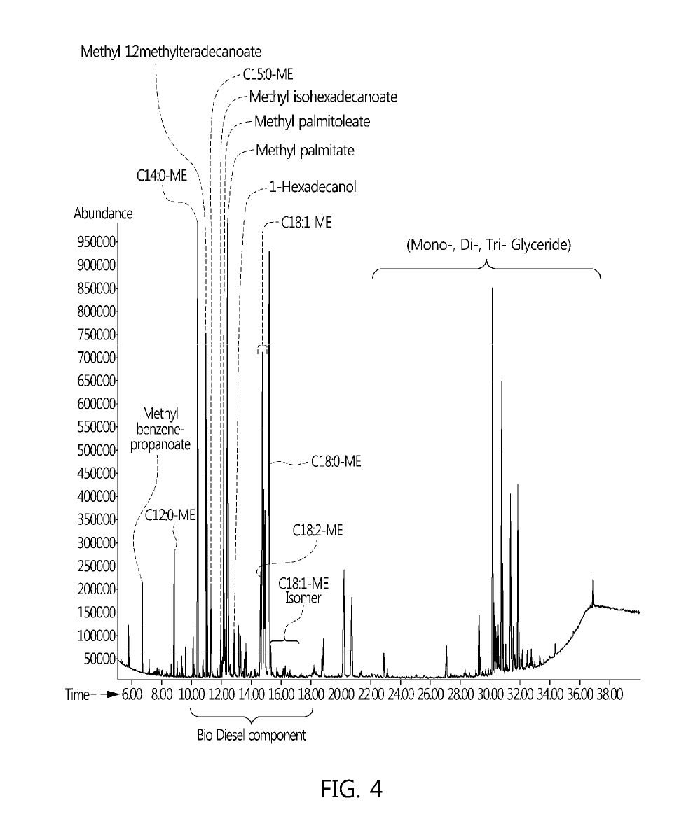

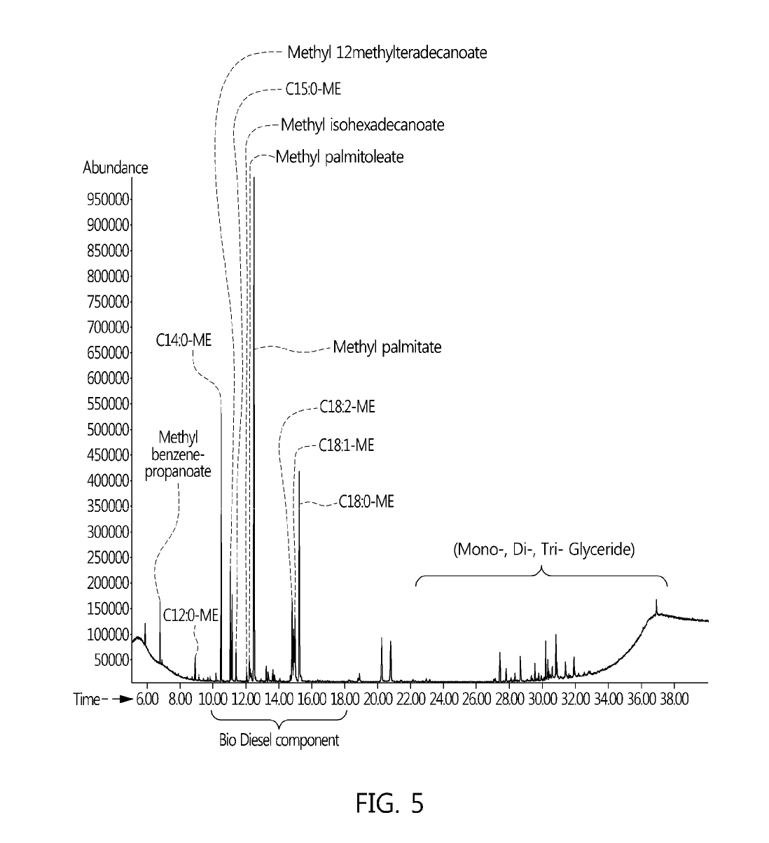

In addition, the use of xylene as in Example 1 afforded biodiesel with a higher content of fatty acid methyl esters than did the use of hexane as in Comparative Example 1. The results are given in FIGS. 4 and 5.

1. A method for producing biodiesel from sewage sludge, including:
(a) heating water-containing sewage sludge and a mixture solution, together with xylene or toluene as a cosolvent, in a reaction tank (30) to induce a transesterification reaction, the mixture solution including methanol or ethanol and an acid catalyst at a volume ratio of 1:0.01 to 1:0.05;
(b) cooling and condensing a mixed steam generated in step (a) to separate respective layers of an aqueous methanol or ethanol solution and the cosolvent, followed by circulating the cosolvent to the reaction tank;
(c) further adding methanol or ethanol to the reaction tank to perform the transesterification reaction to completion, with the concomitant production of biodiesel; and
(d) isolating and recovering the biodiesel from the aqueous methanol or ethanol solution, the cosolvent, and the residual solid.

2. The method of claim 1, wherein the mixture solution is mixed at a volume ratio of 1:0.25 to 1:1 with the cosolvent in step (a), and the transesterification reaction is carried out at 80 to 110° C. for 4 to 8 hrs.

3. The method of claim 1, further including adding a salt to the aqueous methanol or ethanol solution condensed and separated in step (b) to separate the aqueous methanol solution into methanol or ethanol and water, and recycling the methanol or ethanol into the reaction tank.

4. An apparatus for producing biodiesel, including:
a cosolvent reservoir (10);
a solvent reservoir (20);
a reaction tank (30) provided at upper positions thereof with a solvent supply unit (90) for introducing a cosolvent and a solvent, respectively, from the cosolvent reservoir and the solvent reservoir and with a sludge supply unit (80), at a lower portion with a sludge discharge unit (120) for discharging residual sludge from the reactor tank, at a middle position with a biodiesel discharge unit (110), and at a top position with a water steam discharge unit (100);
a condenser connected to the water steam discharge unit (40);
a liquid phase separator (50) for separating the condensed, aqueous methanol or ethanol solution that is released from the condenser;
a solvent recovery unit (41) for recovering the condensed solvent discharged from the condenser;
a solvent recovery tank (60) for storing the solvent recovered by the solvent recovery unit (41); and
a solvent circulating pump (70).

5. The apparatus of claim 4, further including a liquid phase separator (50) to which the aqueous methanol or ethanol solution discharged from the condenser (40) is transferred by a conveyor (51), the liquid phase separator (50) being provided with a potassium carbonate supply pump (52), and at upper positions with a methanol or ethanol recovery unit (53) and a water discharge unit (54).
